(19) United States
(12) Patent Application Publication
(10) Pub. No.: US 2020/0283387 A1
(43) Pub. Date: Sep. 10, 2020

(54) METHOD FOR PREPARING METHIONINE ANALOGUES

(71) Applicant: ADISSEO FRANCE S.A.S., ANTONY (FR)

(72) Inventors: Robert HUET, PARIS (FR); Vivien HENRYON, LYON (FR); Jérôme MONBRUN, CHUZELLES (FR); Etienne AIRIAU, VIENNE (FR); Sylvain AUBRY, CALAIS (FR); Patrick REY, LYON (FR)

(21) Appl. No.: 16/070,762

(22) PCT Filed: Jan. 17, 2017
(86) PCT No.: PCT/FR2017/050096

§ 371 Date: May 6, 2020

(30) Foreign Application Priority Data
Jan. 18, 2016 (FR) 16/50366

(51) Int. Cl. C07D 211/06; C07F 1/04; C07C 321/04

(52) U.S. Cl. CPC C07D 211/06; C07C 321/04; C07F 1/04

(57) ABSTRACT

The invention relates to a method for preparing a compound (I) or one of the salts thereof, and the uses of said method, R¹OOC—C(═X)—CHR²R³(I), wherein X is selected among O; N—R′, wherein R′ is H or a C1-C6 alkyl; and N—OR″ wherein R″ is H, or a C1-C6 alkyl or an alkylaryl; R¹ is H or a C1-C6 alkyl group; R² is H, a C1-C6 alkyl, or an alkylaryl; and R³ is CH₂SR⁴ or CH₂SeR⁴, where R⁴ is H or a C1-C6 alkyl from a compound (II), or one of the salts thereof, R¹OOC—C(═X)—CHR²R⁵ and R⁵ is H or COOR⁶, where R⁶ is H or a C1-C6 alkyl, said method being carried out in the presence of a compound (III) CH₂(Y)(Z). Wherein Y is H; OR⁷, where R⁷ is H, a C1-C6 alkyl or an acyl with formula CO—R⁴; SR⁴ or SeR⁴ where R⁴ matches the preceding definition; or 1 NR⁸R⁹, where R⁸ and R⁹, identical or different, each or together are a C1-C6 alkyl, or an alkylaryl; Z, identical or different to Y, is OR¹⁰ where R¹⁰ is H, a C1-C6 alkyl or CO—R⁴; a cyclic or acyclic N(COR⁴)(COR⁴) group; or NR¹¹R¹², where R¹¹ and R¹², identical or different, each or together are a C1-C6 alkyl or an alkylaryl; wherein Y and Z together are ═O; said method involving an intermediate product (IV) R¹OOC—C(═X)—CHR²—CH₂Z.

### CROSS REFERENCE TO RELATED APPLICATIONS

This application is a U.S. National Stage of PCT Application No. PCT/FR2017/050096 filed on Jan. 17, 2017, which claims priority to French Patent Application No. 16/50366 filed on Jan. 18, 2016, the contents of each are incorporated herein by reference thereto.

The present invention concerns a method for preparing methionine analogues, as well as selenium derivatives of methionine analogues, from abundant and accessible compounds, derived from biomass.

The methionine and the analogues thereof such as 2-hydroxy-4-methylthiobutanoic acid (HMTBA) and 2-oxo-4-methylthiobutanoic acid (KMB), as well as salts, chelates, in particular metal chelates (of Zn, Ca Mn, Mg, Cu, Na . . . ) and the esters of these acids, such as the isopropyl and tert-butyl esters of HMTBA, are widely used in animal nutrition. The selenium derivatives of the methionine and of the hydroxy-analogues thereof are also constituents of major interests in animal nutrition.

Given the continuously growing volumes, consumed worldwide, of these ingredients, it is necessary to develop manufacturing methods from renewable, energy-efficient and non-polluting sources.

The authors have thus developed a method for preparing these compounds from organic acids, the salts or the derivatives thereof, which may be obtained from biomass, in particular by biological transformations such as fermentation methods.

According to the invention, this method allows preparing a compound or a salt thereof, said compound having the formula (I),

R¹OOC—C(═X)—CHR²R³ (I)

wherein X is selected from O; N—R′ where R′ represents H or a C1-C6 alkyl group; and N—OR″ where R″ represents H, a C1-C6 alkyl group or an alkylaryl group;

R¹ represents H or a C1-C6 alkyl group;

R² represents H, a C1-C6 alkyl group, or an alkylaryl group; and

R³ represents CH₂SR⁴ or CH₂SeR⁴ with R⁴ representing H or a C1-C6 alkyl group,

from a compound of formula (II), or a salt thereof,

R¹OOC—C(═X)—CH₂R²R⁵ (II)

wherein R¹, R² and X have the definition above; and

R⁵ represents H or COOR⁶ with R⁶ representing H or a C1-C6 alkyl group.

This method allows manufacturing methionine analogues such as KMB, from acids such as the oxaloacetic acid and the pyruvic acid, in interesting yields for an industrial exploitation, not releasing sub-products in excessive amounts and involving moderate reaction conditions and available reagents. Moreover, these compounds constitute particularly interesting precursors of methionine in its different active forms D,L; D and L and of HMTBA in its different enantiomeric forms D,L; D and L. They may be indeed transformed into said methionine or said HMTBA by simple reduction, for example with hydrides of NaBH₄ type, or by catalytic or biocatalytic hydrogenation, or by racemic or enantioselective biochemical transformation, or by any other method known to those skilled in the art.

More specifically, the method of the invention is carried out in the presence of a compound of formula (III)

CH₂(Y)(Z) (III)

wherein Y represents H; OR⁷ with R⁷ representing H, a C1-C6 alkyl group or an acyl group of formula CO—R⁴ with R⁴ meeting the preceding definition; SR⁴ or SeR⁴ with R⁴ meeting the preceding definition; or NR⁸R⁹, with R⁸ and R⁹, identical or different, representing each or together, a C1-C6 alkyl group, or an alkylaryl group;

Z, identical to or different from Y, represents OR¹⁰ with R¹⁰ representing H, a C1-C6 alkyl group, or CO—R⁴ with R⁴ meeting the preceding definition; a cyclic or acyclic N(COR⁴)(COR⁴) group, with R⁴ meeting the preceding definition; or a NR¹¹R¹² group, with R¹¹ and R¹², identical or different, representing each or together, a C1-C6 alkyl group, or an alkylaryl group;

or Y and Z represent together ═O;

and said method comprises the following steps:

the compound (II) is reacted with the compound (III) to lead to an intermediate having the structure (IV)

R¹OOC—C(═X)—CHR²—CH₂Z (IV)

wherein R¹, R², X and Z have the definition above,

the compound (IV) thus obtained is reacted with R⁴SH or a salt thereof, or R⁴SeH or a salt thereof, with R⁴ meeting the preceding definition, already present in the reaction medium or added during the method,

then, upon completion of the reaction, the compound (I) or a salt thereof is isolated.

As will be described hereinafter, according to a variant of the method, the reaction of the compound (IV) with R⁴SH or a salt thereof, or R⁴SeH or a salt thereof, with R⁴ meeting the preceding definition, already present in the reaction medium or added during the method, leads to a compound having the structure (V)

R¹OOC—C(A)(B)—CHR²—CH₂Z (V)

wherein R¹, R² and Z have the definition above;

A represents OH; HN—R′ where R′ represents H or a C1-C6 alkyl group; or HN—OR″ where R″ represents H, a C1-C6 alkyl group, or an alkylaryl group; and

B represents SR⁴ or SeR⁴ with R⁴ meeting the preceding definition.

Before exposing the invention in more details, some terms used in the text are specified below.

In the formulas defining the compounds and reagents obtained or involved, the term “alkyl” designates a linear or branched monovalent hydrocarbon radical having 1 to 20 carbon atoms, advantageously 1 to 6 carbon atoms, such as methyl, ethyl, propyl, isopropyl, butyl, isobutyl, tert-butyl, pentyl, neopentyl, n-hexyl or a cyclic monovalent hydrocarbon radical having 3 to 20 carbon atoms, advantageously 3 to 6 carbon atoms, such as cyclopropyl, cyclohexyl.

Alkylaryl group means an aryl group comprising 6 to 20 carbon atoms, said aryl group being substituted by at least one alkyl group meeting the definition above.

The invention is detailed hereinafter and its advantageous variants are presented.

The method disclosed above can be implemented according to several approaches.

A first route preferably consists in reacting a compound (II) with the formaldehyde or the paraformaldehyde, in hydrated or non-hydrated form, in a basic medium and in the presence of MeSH or a salt thereof, such as sodium, potassium or calcium salts of methylmercaptan.

A second route includes reacting a compound (II) with a compound (III), said compound (III) being selected from 1-[(methylsulfanyl)methyl]-piperidine, 1-[(methylsulfanyl)methyl]-pyrrolidine and 1-[(methylsulfanyl)methyl]-diethylamine. This second route leads to an intermediate compound which may be isolated or not, which is an object of the present invention. This compound meets the following formula (V):

R¹OOC—C(A)(B)—CHR²—CH₂Z (V)

wherein R¹ represents H or a C1-C6 alkyl group;

R² represents H, a C1-C6 alkyl group, or an alkylaryl group;

A represents OH, HN—R′ where R′ represents H or a C1-C6 alkyl group, or HN—OR″ where R″ represents H or a C1-C6 alkyl group or an alkylaryl group;

B represents SR⁴ or SeR⁴ with R⁴ representing H or a C1-C6 alkyl group;

Z represents OR¹⁰ with R¹⁰ representing H; a C1-C6 alkyl group; a CO—R⁴ group with R⁴ representing H or a C1-C6 alkyl group, a cyclic or acyclic N(COR⁴)(COR⁴) group, with R⁴ meeting the preceding definition; or NR¹¹R¹², with R¹¹ and R¹², identical or different, representing each or together, a C1-C6 alkyl group, or an alkylaryl group.

The second route advantageously brings into contact the oxaloacetic acid or an ester thereof, that is to say a compound of formula (II) in which X represents O, R² represents H and R⁵ represents CO₂R⁶ with R⁶ representing H or a C1-C6 alkyl group, with a compound of formula (III) of the type CH₂(Y)(Z) in which Y and Z represent respectively the group SCH₃ and the group NR¹¹R¹² as defined previously; preferably, the group NR¹¹R¹² represents the piperidinyl group. Therefore, the invention also concerns the compound of formula (V) in which A represents OH, B represents SCH₃, R² represents H and Z represents the piperidinyl group.

According to a third route, it is possible to react the compound (II) with a compound (III) selected from methylenedipiperidine, methylenedipyrrolidine and methylenedi(diethylamine). An intermediate compound of this third route, which can be isolated or not, is also an object of the invention. It meets the following formula (IV):

R¹OOC—C(═X)—CHR²—CH₂Z (IV)

wherein X is selected from O; N—R′ where R′ represents H or a C1-C6 alkyl group; and N—OR″ where R″ represents H, a C1-C6 alkyl group or an aryl group;

R¹ represents H or a C1-C6 alkyl group;

R² represents H, a C1-C6 alkyl group, or an alkylaryl group; and

Z represents NR⁸R⁹, with R⁸ and R⁹, identical or different, representing each or together, a C1-C6 alkyl group, or an alkylaryl group.

As will be illustrated in the examples, the third route advantageously brings into contact the oxaloacetic acid or an ester thereof, that is to say a compound of formula (II) in which X represents O, R² represents H and R⁵ represents CO₂R⁶ with R⁶═H, with a compound of formula (III) in which Y and Z represent respectively the group NR⁸R⁹ and the group NR¹¹R¹² as previously defined. Preferably, at least one of NR⁸R⁹ and NR¹¹R¹², but even better both, represent the same piperidinyl group. Thus, the invention concerns the intermediate compound of formula (IV) in which X represents O, R² represents H and Z represents the piperidinyl group, R¹ being as previously defined, namely H when the compound (II) is the oxaloacetic acid or a C1-C6 alkyl group when the compound (II) is the corresponding ester of the oxaloacetic acid.

Regardless of the retained route, the compound (II) is advantageously selected from oxaloacetic acid and pyruvic acid.

As previously said, the method of the invention allows obtaining different methionine analogues. Particularly, 2-oxo-4-methylthiobutanoic acid (KMB) or a salt thereof, such as the calcium, magnesium, manganese, copper, zinc, sodium or ammonium salts thereof and the selenium analogue thereof or a salt thereof are products of the method of the invention under economically attractive conditions and yields.

Another object of the invention is a method of D,L-methionine, D- or L-methionine, D,L-2-hydroxy-4-methylthiobutanoic acid (HMTBA), or D- or L-HMTBA, from 2-oxo-4-methylthiobutanoic acid (KMB), said method comprising the preparation of KMB according to the method of the invention as defined above then the transformation of KMB thus obtained into methionine or HMTBA, chemically or biologically, by techniques known to those skilled in the art.

The present invention is described in more details by the following examples illustrating the synthesis of KMB from the oxaloacetic acid and the pyruvic acid according to different synthetic routes, all falling within the scope of the invention.

### EXAMPLE 1: PREPARATION OF KMB BY THE FIRST ROUTE IN THE PRESENCE OF NAOH, HCHO AND MESNA

The general scheme of the synthesis is the following:

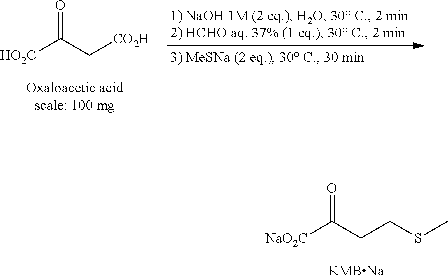

In a reactor, 100 mg of oxaloacetic acid are placed and a solution of NaOH 1 M is added (2 eq.). The reactor is placed at 30° C. and the dissolution of the oxaloacetic acid is immediate. After 2 minutes, the 37% w/w formaldehyde solution is added (1 eq). The stirring is maintained for 2 minutes at 30° C. then the MeSNa (2 eq., 108 mg) is added in one portion and the reaction medium is stirred at 30° C.

A monitoring of the reaction by HPLC-UV (column C18 Hydro-RP) is performed after 10 minutes of contact then every 20 minutes. The best performances were measured after 30 minutes of contact at 30° C. with the following results:
Conversion_{oxaloacetic acid}=100%
Dosed yield in KMB=75%
Selectivity in KMB=75%

### EXAMPLE 2: PREPARATION OF KMB BY THE SECOND ROUTE BY USING THE ACTIVATED THIOMETHYL DERIVATIVE AND THE OXALOACETIC ACID

The general scheme of the synthesis is the following:

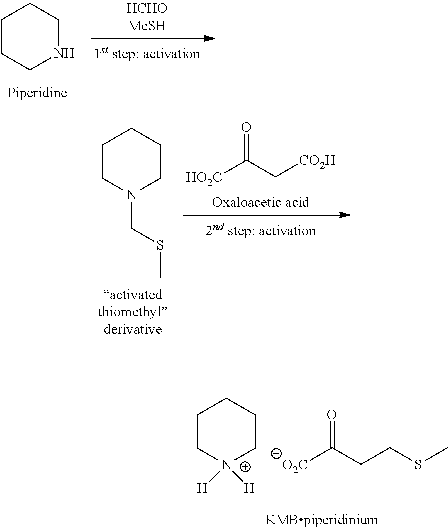

### 1^{st} Step: Synthesis of the Activated Thiomethyl Derivative [Compound (III)]

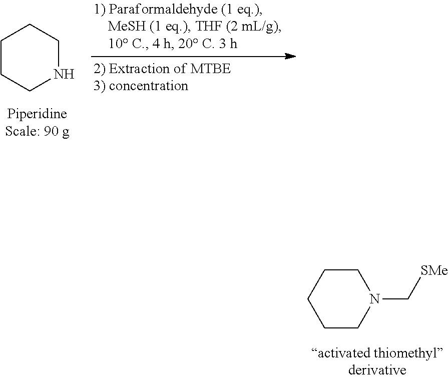 [SMILES: C1CCNCC1 ; CSCN1CCCCC1]

In a 1 L reactor under argon, are introduced successively under stirring at 20° C.:
90.0 g of piperidine
180 ml of THF
34.4 g of paraformaldehyde

The reaction medium is cooled to 10° C. then MeSH is added by bubbling into the reaction medium, at 10° C., until the required amount (1 eq.). The addition is completed in 4 hours then the set temperature is raised to 20° C. The reaction medium is stirred for 3 h at this temperature. A GC-FID control (column Equity-1) indicates that the conversion of piperidine is complete and the RR_{dosed} in “activated thiomethyl” species is of 97%.

180 ml of methyl tert-butyl ether (MTBE) then 180 ml of a NaCl saturated aqueous solution are added to the reaction medium, the two phases are stirred for 5 min and then separated. The organic phase is washed twice with 180 ml of NaCl saturated aqueous solution then dried over Na₂SO₄ and concentrated under reduced pressure (10 mbar, 30° C.). The “activated thiomethyl” derivative is obtained in the form of a colorless liquid without additional purification (150.4 g). The following performances were obtained:
Complete conversion of piperidine
Isolated yield of activated thiomethyl derivative=95%
Titer=94% (dosed by NMR ¹H vs 3,5-dimethylanisole)

### 2^{nd} Step: Synthesis of KMB Piperidinium

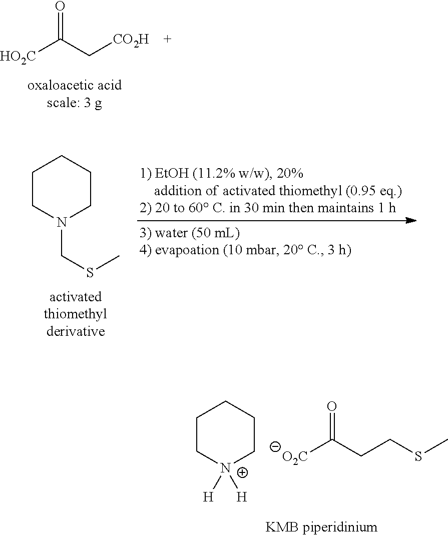

In a 100 mL reactor under argon, provided with a thermostatically controlled bath, 3 g (1 eq.) of oxaloacetic acid and 30 ml of ethanol are introduced. After dissolution at 20° C. (about 1 minute), the activated thiomethyl derivative is added (1 eq.) then, the medium is heated up to 60° C. in 30 minutes then the temperature is maintained for 1 h.

A monitoring of the reaction by HPLC-UV (column C18 Hydro-RP) is performed after 10 minutes of contact then every 20 minutes. The best performances were measured after 1 hour of contact at 60° C. with:
Complete conversion of oxaloacetic acid
Dosed yield in KMB=78%
Selectivity in KMB=78%

The reaction medium is withdrawn then concentrated under reduced pressure (10 mbar, 20° C., 6 h). The KMB piperidinium is obtained in the form of a yellow oil without additional purification.
Isolated yield in KMB piperidinium=60%
Titer=60% (dosed by HPLC vs standard)

### EXAMPLE 3: PREPARATION OF KMB BY THE SECOND ROUTE BY USING THE ACTIVATED THIOMETHYL DERIVATIVE AND THE PYRUVIC ACID

The general scheme of the synthesis is the following:

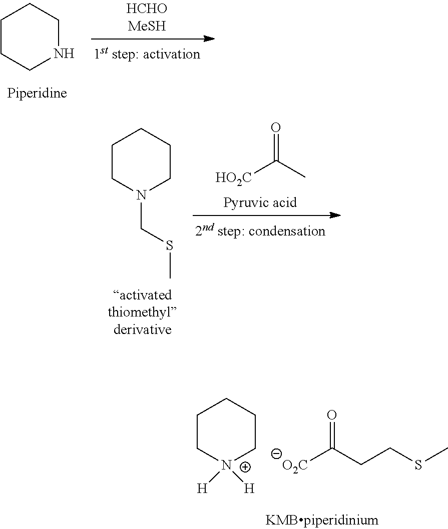

### 1^{st} Step: Synthesis of the Activated Thiomethyl Derivative

It is described in the first step of Example 2.

### 2^{nd} Step: Condensation with the Pyruvic Acid

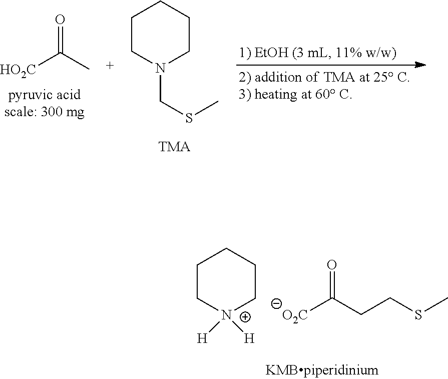

In a Vial, 300 mg (1 eq.) of pyruvic acid and 3 mL of ethanol are introduced. After dissolution at 20° C., the activated thiomethyl derivative (TMA) is added (1 eq.) then the vial is placed in a plate previously heated to 60° C. The reaction medium is stirred at this temperature for 1 hour.

A monitoring of the reaction by HPLC-UV (column C18 Hydro-RP) is performed after 15 minutes of contact then every 15 minutes. The best performances were measured after 15 min of contact at 60° C. with:
Conversion of the pyruvic acid=57%
Yield dosed in KMB=42%
Selectivity in KMB=74%

### EXAMPLE 4: PREPARATION OF KMB BY THE THIRD ROUTE IN A SEQUENCED MANNER VIA METHYLENEDIPIPERIDINE SPECIES

The general scheme of the synthesis is as follows:

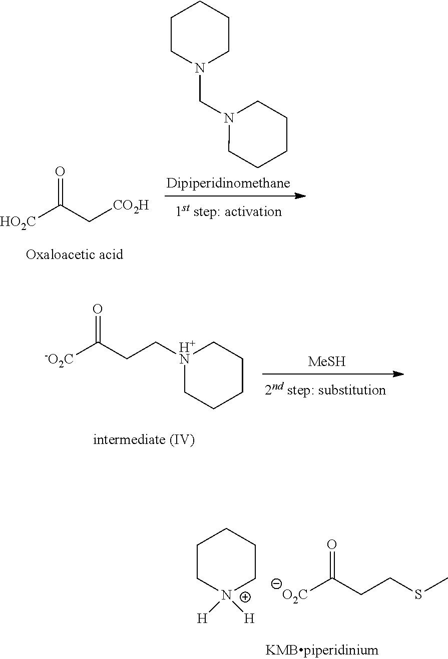

### 1^{st} Step: Synthesis of the Intermediate (IV)

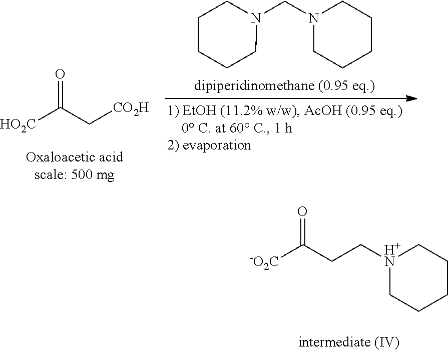

In a 10 mL reactor under argon equipped with a temperature probe, are introduced successively at C.
500 mg of oxaloacetic acid
10-5 mL of EtOH
209 mg of acetic acid

After dissolution of the acid (about 5 minutes of stirring), dipiperidinomethane (0.95 eq.) is added in 50 minutes via a syringe pump. After the end of the addition, the medium is heated to 60° C. in 1 hour then stirred at this 15 temperature for 10 minutes. A control by HPLC-MS (ESI⁺) confirms the formation of the intermediate IV.

The reaction medium is cooled to 20° C. then the solvent is evaporated under reduced pressure (10 mbar, 20° C., 1 h) to lead to (IV) in the form of a pale-yellow oil (1.2 g).
Complete TT_{oxaloacetic acid}
RR_{isolated} of the activated thiomethyl derivative=60%
Titer=35% (dosed by NMR ¹H vs 3,5-dimethylanisole)

### 2^{nd} Step: Synthesis of KMB Piperidinium

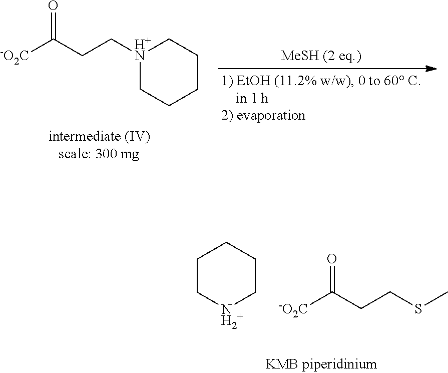

In a 10 mL reactor under argon provided with a temperature probe, are introduced successively under stirring at 0° C.:
300 mg of intermediate (IV)
5 mL of EtOH

Once the reaction medium at a temperature lower than 5° C., the MeSH gas is introduced in 1 hour (via a syringe pump). The reaction medium is then heated to 60° C. in 1 hour then this temperature is maintained for 30 minutes.

An HPLC control indicates a complete transformation of the intermediate and the majority formation of KMB piperidinium.

The reaction medium is withdrawn then concentrated under reduced pressure (10 mbar, 20° C., 1 h). The KMB piperidinium is obtained in the form of a yellow oil without additional purification (260 mg)
Complete TT_{intermediate IV}
RR_{isolated} of the KMB piperidinium=90%
Titer=45% (dosed by HPLC vs standard)

1. A method for preparing a compound or a salt thereof, the compound having the formula (I),
R¹OOC—C(═X)—CHR²R³ (I)
wherein X is O; N—R′ wherein R′ represents H or a C1-C6 alkyl group; or N—OR″ wherein R″ represents H, a C1-C6 alkyl group or an alkylaryl group;
R¹ represents H or a C1-C6 alkyl group;
R² represents H, a C1-C6 alkyl group, or an alkylaryl group; and
R³ represents CH₂SR⁴ or CH₂SeR⁴ with R⁴ representing H or a C1-C6 alkyl group from a compound of formula (II), or a salt thereof,
R¹OOC—C(═X)—CHR²R⁵ (II)
wherein R¹, R² and X are as previously defined; and
R⁵ represents H or COOR⁶ with R⁶ representing H or a C1-C6 alkyl group,
the method being carried out in the presence of a compound of formula (III)
CH₂(Y)(Z) (III)
wherein Y represents H; OR⁷ with R⁷ representing H, a C1-C6 alkyl group or an acyl group of formula CO—R⁴ with R⁴ as previously defined; SR⁴ or SeR⁴ with R⁴ as previously defined; or NR⁸R⁹, with R⁸ and R⁹ are identical or different, each representing a C1-C6 alkyl group; or an alkylaryl group, or R⁸ and R⁹ together represent a C1-C6 alkyl group or an alkylaryl group;
Z is identical to or different from Y and represents OR¹⁰ with R¹⁰ representing H, a C1-C6 alkyl group, or CO—R⁴ with R⁴ as previously defined; a cyclic or acyclic N(COR⁴)(COR⁴) group, with R⁴ as previously defined; or NR¹¹R¹², with R¹¹ and R¹²; are identical or different, each representing a C1-C6 alkyl group or an alkylaryl group, or R¹¹ and R¹² together represent a C1-C6 alkyl group or an alkylaryl group;
or Y and Z together represent ═O;
the method wherein
the compound (II) is reacted with the compound (III) to lead to an intermediate compound of formula (IV)
R¹OOC—C(═X)—CHR²—CH₂Z (IV)
wherein R¹, R², X and Z are as previously defined,
the compound (IV) is reacted with R⁴SH or a salt thereof, or R⁴SeH or a salt thereof, with R⁴ as previously defined, already present in the reaction medium or added during the method, and
upon completion of the reaction, the compound (I) or a salt thereof is isolated.

2. The method according to claim 1, wherein the reaction of the compound (IV) with R⁴SH or a salt thereof or R⁴SeH or a salt thereof, already present in the reaction medium or added during the method, leads to a compound of formula (V)
R¹OOC—C(A)(B)—CHR²—CH₂Z (V)
wherein, R² and Z are as previously defined;
A represents OH, HN—R′ where R′ represents H, a C1-C6 alkyl group, or HN—OR″ where R″ represents H, a C1-C6 alkyl group, or an alkylaryl group; and
B represents SR⁴ or SeR⁴ with R⁴ as previously defined.

3. The method according to claim 1, wherein the compound (II) is reacted with hydrated or non-hydrated formaldehyde or paraformaldehyde, in a basic medium and in the presence of MeSH or a salt thereof.

4. The method according to claim 1, wherein the compound of formula (II) is reacted with the compound of formula (III), wherein the compound of formula (III) is 1-[(methylsulfanyl)methyl]-piperidine, 1-[(methylsulfanyl)methyl]-pyrrolidine, or 1-[(methylsulfanyl)methyl]-diethylamine.

5. The method according to claim 1, wherein the compound of formula (II) is reacted with the compound of formula (III), wherein the compound of formula (III) is methylenedipiperidine, methylenedipyrrolidine, or methylenedi(diethylamine).

6. The method according to claim 1, wherein the compound of formula (II) is oxaloacetic acid or pyruvic acid.

7. The method according to claim 1, wherein 2-oxo-4-methylthiobutanoic acid (KMB) or a salt thereof is obtained.

8. A compound of formula (IV)
R¹OOC—C(═X)—CHR²—CH₂Z (IV)
wherein X is O; N—R′ wherein R′ represents H or a C1-C6 alkyl group; or N—OR″ where R″ represents H, a C1-C6 alkyl group, or an aryl group,
R¹ represents H or a C1-C6 alkyl group;
R² represents H, a C1-C6 alkyl group or an alkylaryl group; and
Z represents OR¹⁰ with R¹⁰ representing H; a C1-C6 alkyl group or CO—R⁴ with R⁴ representing H or a C1-C6 alkyl group, a cyclic or acyclic N(COR⁴)(COR⁴) group, with R⁴ representing H or a C1-C6 alkyl group; or NR¹¹R¹², with R¹¹ and R¹² are identical or different, each representing a C1-C6 alkyl group; or an alkylaryl group, or R¹¹ and R¹² together represent a C1-C6 alkyl group or an alkylaryl group.

9. The compound according to claim 8, wherein X represents O, R² represents H, and Z represents the piperidinyl group.

10. A compound of formula (V):
R¹OOC—C(A)(B)—CHR²—CH₂Z (V)
wherein R¹ represents H or a C1-C6 alkyl group;
R² represents H, a C1-C6 alkyl group or an alkylaryl group;
A represents OH; HN—R′ where R′ represents H or a C1-C6 alkyl group; or HN—OR″ where R″ represents H, a C1-C6 alkyl group, or an alkylaryl group;
B represents SR⁴ or SeR⁴ with R⁴ representing H or a C1-C6 alkyl group; and
Z represents OR¹⁰ with R¹⁰ representing H or a C1-C6 alkyl group or COR⁴ with R⁴ representing H or a C1-C6 alkyl group; or NR¹¹R¹², with R¹¹ and R¹² are; identical or different, each representing a C1-C6 alkyl group; or an alkylaryl group, or R¹¹ and R¹² together represent a C1-C6 alkyl group or an alkylaryl group.

11. The compound according to claim 10, wherein A represents OH, B represents SCH₃, R² represents H, and Z represents a piperidinyl group.

12. The compound of formula (IV) according to claim 9, wherein R¹ represents H.

13. The method according to claim 1, wherein 2-oxo-4-methylthiobutanoic acid (KMB) is prepared, and further comprising
chemically or biologically transforming 2-oxo-4-methylthiobutanoic acid (KMB) to D,L-methionine, D-methionine, L-methionine, D,L-2-hydroxy-4-methylthiobutanoic acid (HMTBA), D-2-hydroxy-4-methylthiobutanoic acid, or L-2-hydroxy-4-methylthiobutanoic acid.

14. The method according to claim 7, wherein the salt is a calcium, sodium, ammonium, manganese, copper, zinc, or magnesium salt of 2-oxo-4-methylthiobutanoic acid (KMB).

15. The method according to claim 2, wherein the compound of formula (II) is oxaloacetic acid or pyruvic acid.

16. The method according to claim 2, wherein 2-oxo-4-methylthiobutanoic acid (KMB) or a salt thereof is obtained.

17. The method according to claim 16, wherein the salt is a calcium, sodium, ammonium, manganese, copper, zinc, or magnesium salt of 2-oxo-4-methylthiobutanoic acid (KMB).

18. The method according to claim 2, wherein 2-oxo-4-methylthiobutanoic acid (KMB) is prepared, and further comprising
chemically or biologically transforming 2-oxo-4-methylthiobutanoic acid (KMB) to D,L-methionine, D-methionine, L-methionine, D,L-2-hydroxy-4-methylthiobutanoic acid (HMTBA), D-2-hydroxy-4-methylthiobutanoic acid, or L-2-hydroxy-4-methylthiobutanoic acid.

19. The compound of formula (V) according to claim 11, wherein R¹ represents H.
